(19) United States
(12) Patent Application Publication
(10) Pub. No.: US 2023/0072401 A1
(43) Pub. Date: Mar. 9, 2023

(54) RESPIRATORY MONITORING DEVICE

(71) Applicant: BREATHEOX LIMITED, Oxford (GB)

(72) Inventors: John Paul Moszynski, Oxfordshire (GB); William Ilhoon Do, Surrey (GB); Mikesh Udani, Oxfordshire (GB)

(21) Appl. No.: 17/760,484

(22) PCT Filed: Feb. 10, 2021
(86) PCT No.: PCT/GB2021/050309

§ 371 Date: Aug. 10, 2022

(30) Foreign Application Priority Data
Feb. 11, 2020 (GB) 2001838.8

(51) Int. Cl. A61B 5/08; A61B 5/00

(52) U.S. Cl. CPC A61B 5/0803; A61B 5/7475; A61B 5/7455; A61B 5/742; A61B 2560/0242; A61B 2560/0266; A61B 2562/04; A61B 7/003

(57) ABSTRACT

There is provided a respiratory monitoring device (100) comprising one or more respiratory-related sensors (110) and a first circuit board comprising a controller. The one or more respiratory-related sensors are separated from and in communication the first circuit board. There is also provided a respiratory monitoring device comprising a controller, one or more respiratory-related sensor and an input means (114). The input means is arranged to trigger the controller to suspend sensing by at least one of the one or more respiratory-related sensors for one or more predetermined time periods. There is also provided a respiratory monitoring device having a housing comprising a front surface (102) at which one or more respiratory-related sensors are arranged, and a back surface (120), wherein the front surface is arranged to indicate an intended orientation to a user of the respiratory monitoring device.

### FIELD OF INVENTION

The present invention relates to respiratory monitoring devices, and more particularly respiratory monitoring devices configured to measure respiratory related data of a patient.

### BACKGROUND

Respiratory illnesses, such as asthma, can present serious health risks to sufferers. Respiratory monitoring of patients suffering from such respiratory illnesses can be beneficial for both research purposes into respiratory illness, and for monitoring to the respiratory health of patients with a respiratory illness.

An object of the invention is to provide a respiratory monitoring device that can provide useful, objective, data for researchers and patients in the field of respiratory illnesses.

### SUMMARY OF INVENTION

In an aspect there is provided a respiratory monitoring device configured to measure respiratory related data, the respiratory monitoring device comprising: one or more respiratory-related sensors; a first circuit board comprising a controller of the respiratory monitoring device; wherein the one or more respiratory-related sensors are separated from and in communication the first circuit board.

In this way, the separation between the respiratory-related sensors and the first circuit board inhibits heat generated by components of the controller affecting measurements of the respiratory-related sensors.

Preferably, the one or more respiratory-related sensors are components of a second circuit board, wherein the first circuit board is connected to the second circuit board by one or more spacers in a stacked arrangement, the one or more spacers defining a separation space between the first circuit board and the second circuit board.

In this way, the respiratory-related sensors are arranged on a different circuit board to the controller thereby inhibiting heat generated by the controller components affecting measurements of the respiratory-related sensors.

Preferably, the one or more spacers are columnar.

Preferably, components of the controller are mounted on the first circuit board to face away from the second circuit board; and the respiratory-related sensors are mounted the second circuit board to face away from the first circuit board.

In this way, the respiratory-related sensors are not directed toward the controller components and the controller components are not directed toward the respiratory-related sensors; this inhibits heat generated by the controller components affect measurements of the respiratory-related sensors.

Preferably, one or more substantially heat-generating components of the controller are arranged at a first end portion of the first circuit board, and the respiratory-related sensors are arranged at a second end portion of the second circuit board, the second end portion being an opposing end portion to the first end portion such that the one or more respiratory-related sensors do not directly overlay the one or more substantially heat-generating components.

In this way, a distal separation is provided between the substantially heat-generating components of the controller and the respiratory-related sensors, thereby inhibiting heat generated by the substantially heat-generating components affecting measurements of the respiratory-related sensors.

Preferably, the one or more heat-generating components comprises a processor.

Preferably, a heat insulating material is arranged between the first circuit board and the second circuit board in the separation space.

In this way, a further improvement to the inhibition of heat generated by the components of the controller affecting the measurements of the respiratory-related sensors can be made.

In another aspect there is provided a respiratory monitoring device configured to measure respiratory related data, the respiratory monitoring device comprising: a controller; one or more respiratory-related sensors; and an input means; wherein the input means is arranged to trigger the controller to suspend sensing by at least one of the one or more respiratory-related sensors for one or more predetermined time periods.

This is advantageous as it gives the user control as to when they do and do not want the measurements and data recording to take place. This is also advantageous as if, for example, a second person enters the room in which the respiratory monitoring device is actively recording data the measurements can be suspended such that the second person's presence, in the vicinity of the device, does not interfere with the measurements. In this way, the quality and usefulness of the recorded data is improved.

Preferably, at least one predetermined time period of the one or more predetermined time periods is a fixed time period.

In this way, the measurement and recording of data can be suspended for a given amount of time, to allow the second person to carry out their task(s) in the vicinity of the device for example, and then resumed. This improves the quality and usefulness of the recorded data as no data is recorded when the second person is present, for example.

Preferably, at least one predetermined period of the one or more predetermined time periods is a time period between the respiratory-related sensors being suspended and a scheduled end time for a respiratory data measurement session during which the suspension is triggered.

In this way, unnecessary data is not recorded, and only useful data is recorded. This improves the quality and usefulness of the recorded data.

Preferably, the input means comprises a user-operable button.

In this way, a user of the device can suspend the sensing in a simple, user-friendly manner.

Preferably, the user-operable button is actuatable in a first manner to trigger the device to suspend sensing for a first predetermined time period of the one or more predetermined time periods, and in a second manner to trigger the device to suspend sensing for a second predetermined time period of the one or more predetermined time periods, the second predetermined time period different to the first time period.

In this way, the user has the option of selecting a predetermined time period for the suspension, from multiple different predetermined time periods, with a single button. This is useful as it allows the user to suspend the sensing for the most suitable time period, thereby not needing to suspend the sensing for a time period unnecessarily longer than required, or shorter than required. This further contributes to improving the quality and usefulness of the recorded data.

Preferably, in the first manner the user-operable button is actuated for a first actuation time, and in the second manner the user-operable button is actuated for a second actuation time.

In this way, the user can select the predetermined time period in a user-friendly manner.

Preferably, the user-operable button is depressable in two stages such in the first manner the user-operable button is partially depressed, and in the second manner the user-operable button is fully depressed.

In this way, the user can select the predetermined time period in a user-friendly manner.

Preferably, the user-operable button is arranged to provide a haptic feedback indicating that the user-operable button has been partially depressed and fully depressed.

In this way, the user can clearly identify which predetermined time period they have selected. This decreases the likelihood of the user (inadvertently) selecting a less suitable predetermined time period for the suspension, and therefore improves the quality and usefulness of the recorded data.

Preferably, the respiratory monitoring device further comprises an indicator arranged to indicate when the device has been triggered to suspend sensing.

In this way, the user can identify that the suspension has been triggered. As such, the user can identify whether or not or a suspension has been triggered, and if not they can trigger a suspension. This improves the quality and the usefulness of the recorded data as it helps to ensure that a suspension is triggered when required.

Preferably, the indicator is arranged to indicate information relating to the predetermined time period for which sensing is suspended.

In this way, the user can identify which predetermined time period has been triggered. This improves the quality of usefulness of the data as it allows for a corrective action to be taken if a different predetermined time period is required for the suspension.

Preferably, the indicator comprises a light source.

In this way, the information relating to the predetermined time period is conveyed in a clear and user-friendly manner. This can reduce the likelihood of operational errors by the user, thereby improving the quality and usefulness of the recorded data.

Preferably, the light source is arranged at a different side of the respiratory monitoring device to a side at which the one or more respiratory-related sensors are arranged.

In this way, the light source is not directed toward the patient, and therefore is less likely to disturb the patient.

Preferably, the respiratory monitoring device further comprises an ambient light sensor, and wherein a brightness of the light source is adjusted in accordance with an ambient light level detected by the ambient light sensor.

In this way, when the ambient light level is high, i.e. the room is relatively brightly lit, the brightness of the operational information indicator light source can be set to a higher level so that the light emitted is clearly visible to a user. When the ambient light level is low, i.e. the room is relatively dark, the brightness of the operational information indicator light source can be set to a lower level so that the light emitted is less likely to disturb the sleep of a patient whilst still being visible by a separate user to the device. This improves the usability of the device, and decreases the likelihood of disturbing the patient, thereby contributing to improving the quality and usefulness of the recorded data.

In another aspect there is provided a respiratory monitoring method at a respiratory monitoring device, the method comprising: measuring, by one or more respiratory-related sensors of the respiratory monitoring device, respiratory-related data relating to a person; detecting, by a controller of the respiratory monitoring device, a trigger to suspend sensing by at least one of the one or more respiratory-related sensors for a predetermined time period; suspending, by the controller of the respiratory monitoring device, measuring respiratory-related data relating to the person by at least one of the one or more respiratory-related sensors for the predetermined time period.

This is advantageous as if, for example, a second person enters the room in which the respiratory monitoring device is actively recording data the measurements can be suspended such that the second person's presence, in the vicinity of the device, does not interfere with the measurements. In this way, the quality and usefulness of the recorded data is improved.

In another aspect there is provided a non-transitory computer-readable medium storing instructions that, when executed by one or more processors of a respiratory monitoring device, cause the one or more processors to carry out the steps of: measuring, by one or more respiratory-related sensors of the respiratory monitoring device, respiratory-related data relating to a person; detecting, by a controller of the respiratory monitoring device, a trigger to suspend sensing by at least one of the one or more respiratory-related sensors for a predetermined time period; suspending, by the controller of the respiratory monitoring device, measuring respiratory-related data relating to the person by at least one of the one or more respiratory-related sensors for the predetermined time period.

This is advantageous as if, for example, a second person enters the room in which the respiratory monitoring device is actively recording data the measurements can be suspended such that the second person's presence, in the vicinity of the device, does not interfere with the measurements. In this way, the quality and usefulness of the recorded data is improved.

In another aspect there is provided a respiratory monitoring device configured to measure respiratory related data, the respiratory monitoring device having a housing comprising a front surface at which one or more respiratory-related sensors are arranged, and a back surface, wherein the front surface is arranged to indicate an intended orientation to a user of the respiratory monitoring device.

In this way, the user of the device is guided to direct the respiratory-related sensors toward the patient so as to measure and record high quality, useful data.

Preferably, the front surface is moulded so as to include a recessed region in which at least one of the one or more respiratory-related sensors are arranged.

The recessed region aids the user in identifying the front surface of the device, and the respiratory-related sensors, so that they can be directed toward the patient. This contributes to measuring and recording high quality, useful data.

Preferably, one or more of the back surface and a side surface connecting the front surface and the back surface comprise one or more ventilation holes.

In this way, an airflow can be achieved to inhibit the effect of substantially heat-generating components within the device causing a localised heating that might interfere with measurements by the respiratory-related sensors. Consequently, higher quality, useful data can be measured and recorded.

Preferably, the respiratory monitoring device further comprises an indicator arranged at a side surface of the housing, wherein the side surface connects the front surface and the back surface.

In this way, when the respiratory-related sensors are directed toward the patient (i.e. during a measurement session) the indicator is not directed toward the patient, and is therefore less likely to disturb the patient. This improves the usability of the device, and also allows for higher quality data to be recorded from an undisturbed patient.

Preferably, the respiratory monitoring device further comprises one or more data ports arranged at at least one of the back surface or a side surface of the housing connecting the front surface and the back surface.

The data ports allow for the measured data to be exported from the device. Moreover, arranging the data ports at a back or side surface helps guide the user as to which surface is the front surface at which the respiratory-related sensors are arranged. This contributes to aiding the user in directing the respiratory-related sensors toward the patient so as to measure and record high quality, useful data.

Preferably, the respiratory monitoring device further comprises a power supply input arranged at the back surface of the respiratory monitoring device.

Arranging the power supply input at the back of the device provides stability. Moreover, arranging the power supply input at the back surface helps guide the user as to which surface is the front surface at which the respiratory-related sensors are arranged. This contributes to aiding the user in directing the respiratory-related sensors toward the patient so as to measure and record high quality, useful data.

Preferably, the respiratory monitoring device further comprises a user input means arranged on a side surface of the housing, wherein the side surface connects the front surface and the back surface.

In this way, the user input means is easily accessible to the user without interfering with the respiratory-monitoring sensors. This improves the usability of the device.

Preferably, the respiratory monitoring device further comprises a device diagnostics indicator arranged to indicate diagnostic information relating to an operating state of the respiratory monitoring device to a user of the respiratory monitoring device.

In this way, a user can identify a fault condition of the device for example.

Preferably, the diagnostic indicator is arranged at a side surface of the housing, wherein the side surface connects the front surface and the back surface.

Preferably, the side surface at which the device diagnostics indicator is arranged is a substantially bottom-most surface of the housing when the respiratory monitoring device is in normal use.

As a user will only need to use the device diagnostic indicator occasionally, placing it on the bottom surface of the device reduces the likelihood the user misunderstanding the purpose and confusing it with a respiratory-related sensor or operational indicator of the device. This improves the usability of the device, and helps to ensure that the user directs the respiratory-related sensors toward the patient by making it clear which surface comprises the respiratory-related sensors. This contributes to the measurement and recording of high quality, useful data

Preferably, the one or more respiratory-related sensors are arranged to measure at least one of: movement proximal to the respiratory monitoring device, sound proximal to the respiratory monitoring device, or environmental data proximal to the respiratory monitoring device.

In this way, data relating to the movement of the patient (such as the rise and fall of the patient's chest when breathing), sound data of the patient (such as breathing, coughing or wheezing), and environmental data in the vicinity of the patient can be recorded simultaneously and combined to provide extensive useful data, for an accurate overall analysis, relating to the patients respiratory condition.

Preferably, the respiratory monitoring device comprises at least one of: a radar sensor to measure movement proximal to the respiratory monitoring device; a microphone to measure acoustic data proximal to the respiratory monitoring device; a temperature sensor arranged to measure the ambient temperature proximal to the respiratory monitoring device; or a humidity sensor arranged to measure humidity proximal to the respiratory monitoring device.

### BRIEF DESCRIPTION OF THE DRAWINGS

Embodiments of the invention are now described, by way of example, with reference to the drawings, in which:

FIG. 1 is a perspective diagram of a front view of a respiratory monitoring device;

FIG. 2 is a perspective diagram of a rear view of the respiratory monitoring device;

FIG. 3 is a diagram of a side view of the respiratory monitoring device;

FIG. 4 is a perspective diagram of a bottom view of the respiratory monitoring device;

FIG. 5 is a perspective diagram of a top view of the respiratory monitoring device;

FIG. 6A is a perspective diagram of circuit boards of the respiratory monitoring device;

FIG. 6B is a perspective, semi-exploded, diagram of the circuit boards and housing of the respiratory monitoring device;

FIG. 7 is a conceptual diagram of a stacked arrangement of circuit boards for the respiratory monitoring device;

FIG. 8 is a semi-exploded diagram of a bottom view of the circuit boards and housing of the respiratory monitoring device; and

FIG. 9 is flow chart of a respiratory monitoring method.

### DETAILED DESCRIPTION

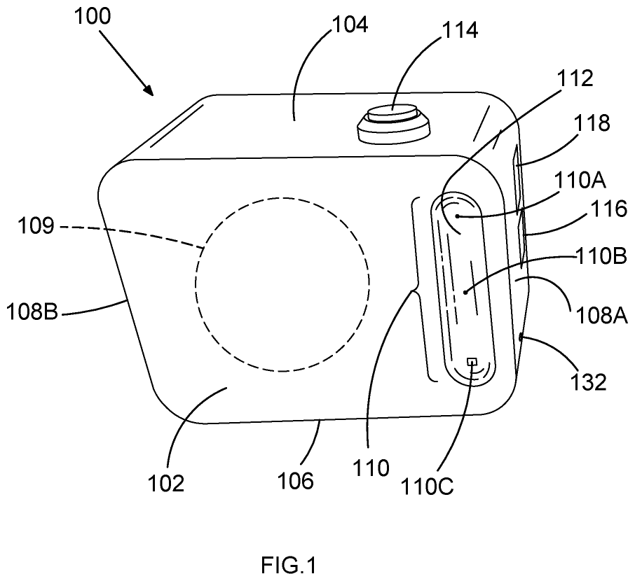

FIGS. 1 to 8 show a respiratory monitoring device 100 according to an embodiment of the present disclosure. The respiratory monitoring device 100 is configured to measure respiratory related data of a patient or person(s) subject to respiratory monitoring. Respiratory-related data can include data relating to the physiology of the patient as well as the environment in which the patient is located. For example, the respiratory monitoring device 100 can be used to record respiratory data for an asthma sufferer as they sleep. This data can then be used for both diagnostic purposes, such as identifying early warning signs for future asthma attacks, and for research purposes.

The respiratory monitoring device 100 includes a housing having a front surface 102, a back surface 120, and side surfaces 104, 106, 108A, 108B connecting the front surface 102 and back surface 120.

The respiratory monitoring device 100 has a back surface 120 opposing the front surface 102. The back surface 120 is in connection to the front surface by side surface portions 104, 106, 108A, 108B. In the example of FIG. 1 these side surface portions include a left-most side surface 108B, a right-most side surface 108A, and top side surface 104 and a bottom side surface 106. The left-most side surface 108B opposes the right-most side surface 108A, and the top side surface 104 opposes the bottom side surface 106.

In an example, the housing can be made from moulded material such as plastic. In the example of FIGS. 1 to 8, the front surface 102 and side surfaces 104, 106, 108A, 108B are formed from a single piece of moulded plastic, whilst the back surface 120 is formed as a separate panel attachable (for example by a plurality of screws 128) to this to form an enclosed volume within the housing.

The respiratory monitoring device 100 has respiratory-related sensors arranged at the front surface 102. The respiratory-related sensors can be used to monitor physiological data of the patient, and environmental data relating to the environment proximal to the patient for respiratory analysis. In use, the respiratory monitoring device 100 is placed proximal to a patient when sleeping, for example on a nightstand, such that the respiratory-related sensors are directed toward the patient. In other words, the respiratory monitoring device 100 is arranged to be placed next to and approximately level with the patient when sleeping. In this way the respiratory-related sensors can record measurements of respiratory data relating to the patient's breathing and the environment around the patient whilst the patient sleeps.

In the example of FIG. 1, the respiratory monitoring device 100 has respiratory-related sensors, the respiratory-related sensors include a motion sensor (such as a radar sensor) mounted behind the front surface 102, and further respiratory-related sensors 110 mounted behind openings in the front surface 102. The radar sensor can be arranged behind the front surface at a substantially central location 109. The respiratory-related sensors 110 mounted behind the openings can include environmental sensor(s) (including at least one of a temperature sensor, humidity sensor, pressure sensor, and volatile organic compound sensor or any other suitable environmental sensor) 110C, an ambient light intensity sensor 1108 and an acoustic sensor (such as a microphone) 110A. The radar sensor is arranged to measure patient movement, the environmental sensor(s) 110C are arranged to measure environmental conditions such as the ambient temperature proximal to the respiratory monitoring device 100 (i.e. the temperature around the patient) and the microphone 110A is arranged to record acoustic data or sound emitted by the patient (i.e. the respiratory sounds relating to the patient breathing). The ambient light sensor 1108 is used to measure ambient light conditions. The skilled person will, however, readily understand that any suitable combination of the aforementioned sensors, and any other suitable number and/or type of sensors for measuring patient respiratory data including physiological and environmental data can be used as appropriate, in any order.

In the example of FIG. 1, the respiratory-related sensors 110 (other than the radar sensor) are arranged in a vertical line on a rightward side portion of the front surface 102 of the respiratory monitoring device 100. The respiratory-related sensors can be arranged in a recessed portion 112 of the front surface. The recessed portion 112 can be considered as a concave (or inwardly curved) area, inward to the device 100 in the front surface 102, relative to the non-recessed portion of the front surface 102. Openings are provided in the front surface 102 behind which the respiratory-related sensors 110 are respectively mounted on a circuit board 202 housed within the housing, as will be described subsequently. That is, the respiratory-related sensors 110 are mounted on a circuit board 202 inside the device 100, and exposed through openings in the front surface 102 of the device 100. The radar sensor is arranged behind the flat portion of the front surface (i.e. an area 109 in the non-recessed portion of the front surface 102). Arranging the radar sensor behind the front surface can allow for a minimum clearance distance to be met between the user of the device 100 and the radar sensor.

Recessing a portion 112 of the front surface 102, that is making it thinner than the remainder of the front surface 102, allows for the opening or port through the front surface 102 behind which the respiratory-sensors 110 are arranged to be as short as possible. This is particularly advantageous for components such as the microphone and ambient light sensor as a longer port (i.e. a deeper opening through thicker material) can negatively affect the quality of the measured data; a shorter port (i.e. an opening through thinner material) can address this issue. Only recessing a portion 112, rather than making the entire front surface 102 thinner, allows for the remainder of the front surface 102 to be thicker, thereby providing structural integrity for the device 100.

The environmental sensor(s) 110C, particularly a temperature sensor, is preferably arranged as the bottom-most sensor of the respiratory-related sensors 110 in the recessed portion 112. As heat rises, positioning the environmental sensor toward the bottom of the front surface 102 helps to mitigate effects of localised heating from the heat-generating components (such as processors) of the device 100 itself which could affect the reliability of ambient temperature measurements.

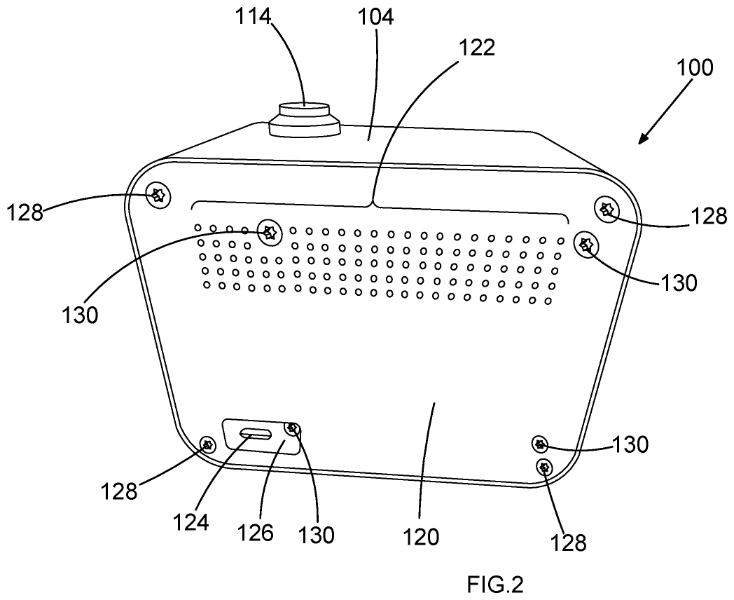

FIG. 2 shows the back surface 120 of the respiratory monitoring device 100. The back surface 120 has a power input port 124 such as a micro USB port. In another example the power input can be hardwired to the device 100. The power input port 124, in the example of FIG. 2, can be arranged in a recessed portion 126 of the rear surface; the recessed portion 126 can be considered a concave area, inward to the device 100 from the back surface 120, relative to the non-recessed portion of the back surface 120. This recess can inhibit damage to the power input port 124. The skilled person will readily understand that the power input port 124 could also be included in any other suitable surface of the device 100. The back surface 120 further includes a plurality of ventilation holes 122, at an upper portion of the back surface 120, arranged to provide and airflow between the internal volume of the device 100 and the external region surrounding the device 100. The airflow provided by the ventilation holes 122 inhibits the internal volume of the device 100 heating substantially due to heat emitted during the operation of the computing components housed therein. This is advantageous in that it minimises the effects of internal heating within the device 100, caused by the computing components of the device 100, interfering with the measured temperature.

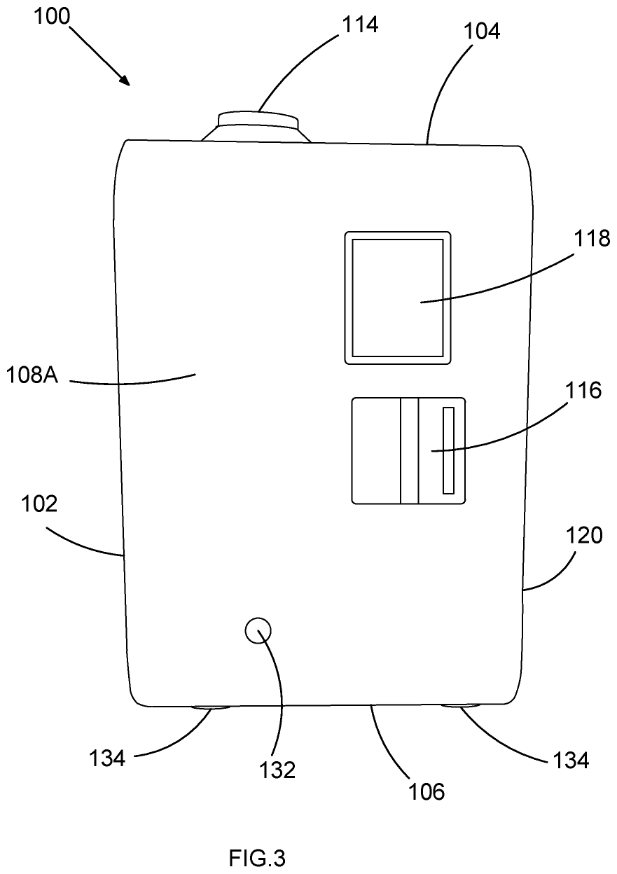

FIG. 3 shows the right-most side surface 108A of the respiratory monitoring device 100. The right-most side surface 108 can include one or more data ports 116, 118, such as a USB port 116 or an Ethernet port 118. The skilled person will however readily understand that the one or more data ports need not be in the right-most side surface 108A of the device 100 and can instead be arranged in any suitable surface of the device 100. Although the figures depict one USB port 116 and one Ethernet port 118, the skilled person will readily understand that any suitable number of data ports can be used, including zero data ports, with the data ports being of any suitable type. For example, the device may include only one data port, said data port being a USB port. In some examples, a data port, such as an Ethernet port, may be included within the device 100 but not accessible to the end user; this data port may be used by a developer or technician when setting up or updating the device. In some examples, the respiratory monitoring device 100 may also include means for a wireless interface between the respiratory monitoring device 100 and an external device, for example through a Bluetooth or WiFi interface. The right-most side surface 108A of the device 100 further includes an indicator 132 arranged to convey operational information to a user of the device 100. The user of the device 100 may be the patient themselves, or a separate user operating the device 100 for the patient. In the example FIG. 1 the indicator 132 is a light source 132, such as a light emitting diode (LED), although other suitable indicators can also be used. This operational indicator 132 will be discussed in more detail subsequently.

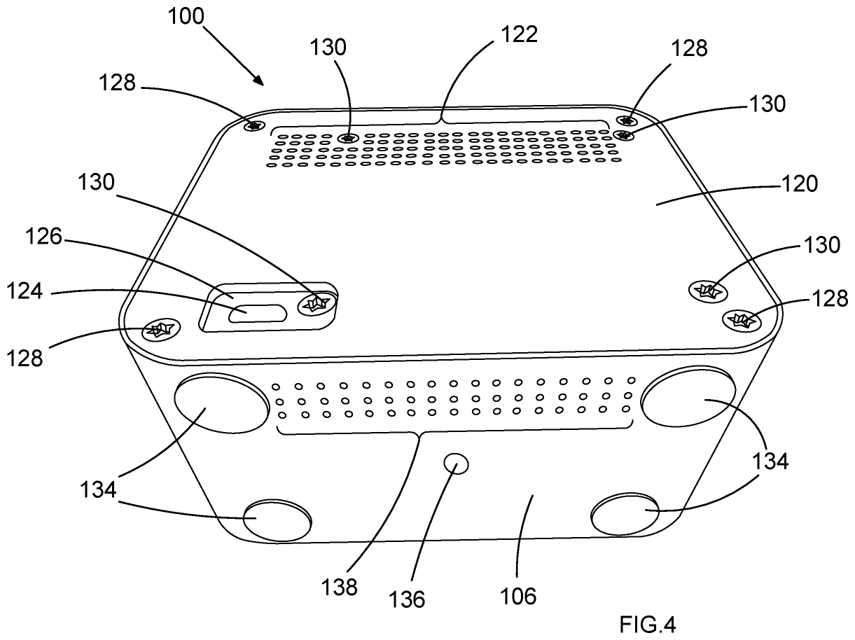

FIG. 4 shows the bottom side surface 106 of the respiratory monitoring device 100. The bottom side surface 106 of the respiratory monitoring device 100 includes a plurality of ventilation holes 138 arranged to provide and airflow between the internal volume of the device 100 and the external region surrounding the device 100. These ventilation holes 138 further contribute to minimising the effects of internal heating within the device 100, caused by the computing components of the device 100, interfering with the measured temperature by the temperature sensor.

The bottom side surface 106 further includes a device diagnostic indicator 136. In an example the device diagnostic indicator 136 is a light source such as an LED. The device diagnostic indicator 136 can, however, be any other suitable type of indicator. The device diagnostic indicator 136 is used to provide the user with an indication of diagnostic information such as a fault condition that the device 100 is experiencing. This information can be conveyed by flashing the diagnostic indicator in a pattern or at a frequency, or illuminating the indicator in a colour, associated with a specific fault condition. Positioning the device diagnostic indicator 136 on the bottom surface is advantageous as a user will not frequently be required to see this indicator. As such, positioning it on the bottom of the device prevents the user confusing the device diagnostic indicator 136 with the operational indicator 132; it also reduces the likelihood of any illumination of the diagnostic indicator disturbing the sleep of the patient. In some examples, the operational indicator 132 may be used to indicate to the user to check the diagnostic indicator 136.

A plurality of feet 134 can be attached to the bottom side surface 106 of the respiratory monitoring device 100. These feet 134 are arranged to create a separation between the bottom side surface 106 and a surface upon which the device 100 is standing when in use. This allows for air flow from the ventilation holes 138. In an example, the feet are rubber pads 134. This is advantageous in that, in addition to providing the separation between the bottom side surface 106 and the surface upon which the device 100 is standing, the pads can also inhibit the device 100 from sliding on such a surface.

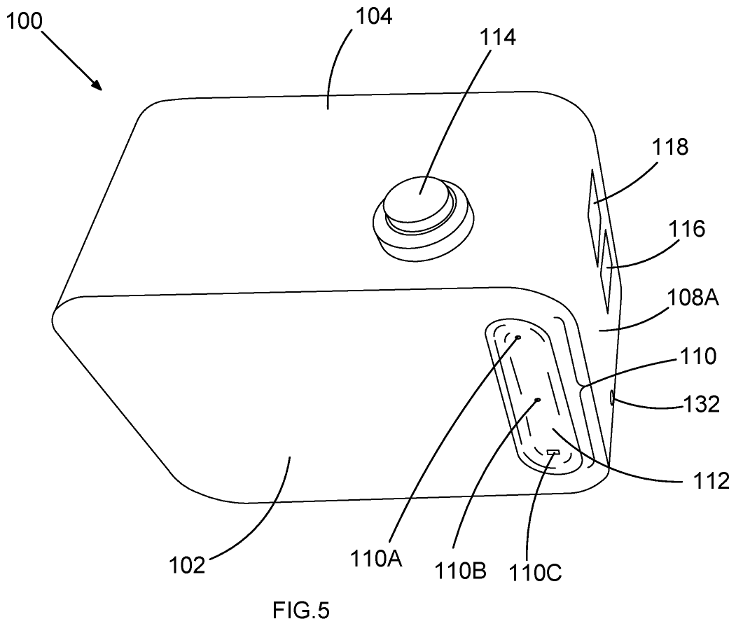

The top side surface 104 of the respiratory monitoring device 100 can be most clearly seen in FIG. 5. A user input means 114 is provided at a rightward side portion of the top side surface 104, in the example of FIG. 5 the user input means is a pushbutton 114. The skilled person will readily understand that other suitable user input means, such as a capacitive button can be used in place of the pushbutton. The skilled person will also understand that the user input means need not be provided at the rightward side portion of the top surface, and could instead be arranged on any other suitable surface. The operation of the user input means will be discussed in more detail subsequently.

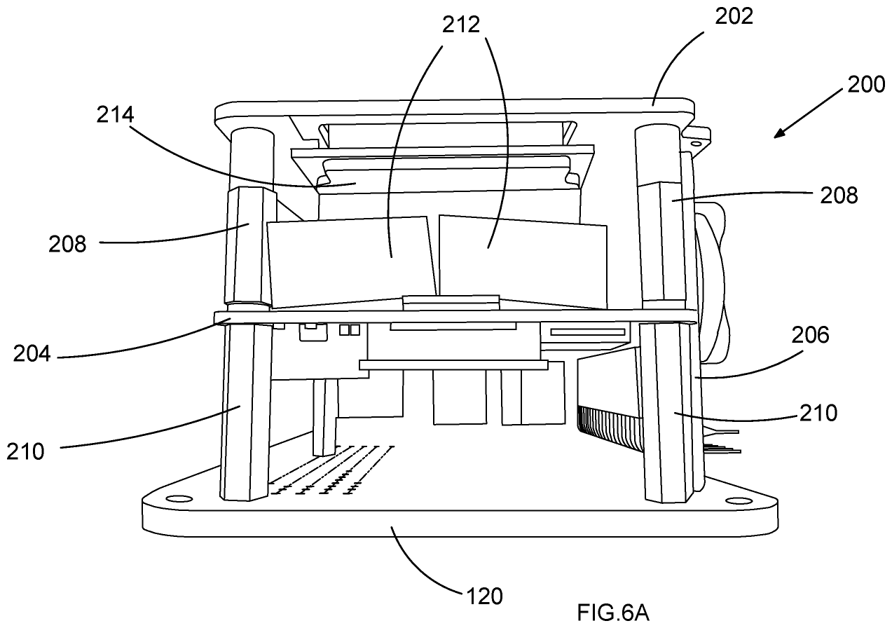

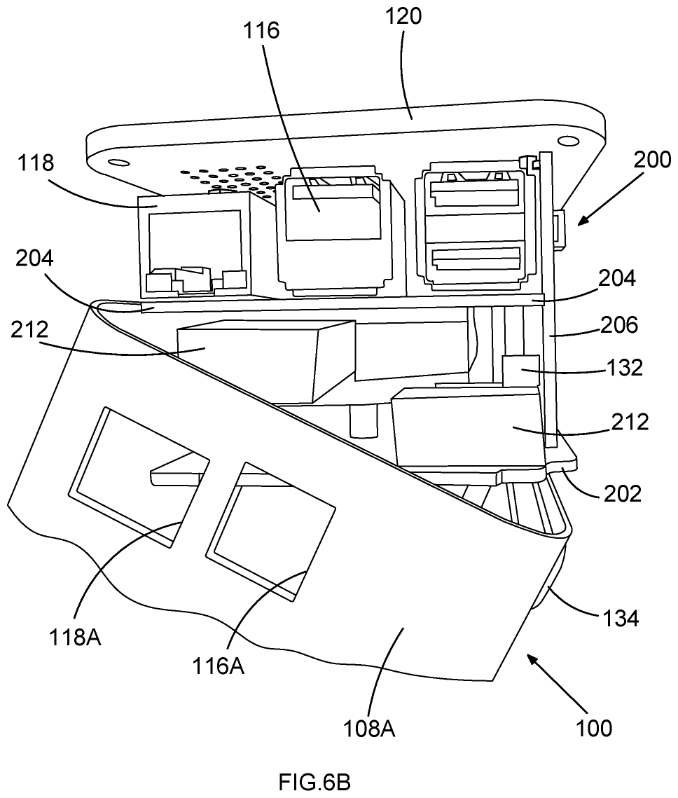

FIGS. 6a and 6b show the internal structure of the device respiratory monitoring device 100, and the arrangement of the electronic components of the respiratory monitoring device 100 housed within the housing. FIG. 6a shows a view from the left-most side of the circuit boards 202, 204, 206; that is, the left-most side being the side of the circuit boards nearest to the left-most side surface of the housing when the circuit boards are contained within the housing. FIG. 6b shows a view from the right-most side of the circuit boards; that is, right-most side being the side of the circuit boards nearest to the right-most side surface of the housing when the circuit board are contained within the housing. A first circuit board, or control board, 204 is used is used to control the operation of the device 100. That is, a controller or control electronics are mounted on the first circuit board or control board 204. The respiratory-related sensors are mounted on a second circuit board, or sensor board, 202 which is in turn connected to the control board 204 such that the controller controls the operation of the respiratory-related sensors and data measurements collected by the respiratory-related sensors can be fed to the controller. The control board 204 can include storage media and one or more processors programmed to execute measurement routines using the respiratory-related sensors. Respiratory-related data measurements, measured by the respiratory-related sensors, are storable in the storage media of the controller. In an example, the control board 204 can be based on a Raspberry Pi, or a specifically designed control board, or by any other suitable approach for arranging control electronics.

The one or more data ports 116, 118 (such as a USB port 116, an Ethernet port 118 etc.) are mounted on the control board 204 such that they are aligned with respective openings 116A, 118A in the side surface 108A of the housing. In this way, a data connection can be achieved between the control board 204 and an external device without needing to physically open the housing. The data ports 116, 118 can be used to export respiratory-related sensor data from the storage media, or to install updates to the controller. For example, a USB stick can be inserted into a USB data port 116, or a USB cable connected to an external device such as a computer or smartphone can be inserted into the USB data port 116.

The power input port 124 can be mounted on the control board 204 such that it is aligned with a respective opening in the back surface 120 of the housing (as in the example of FIG. 2, or in a side surface as appropriate in other embodiments). In the example of FIG. 5, the power input port 124 is a micro USB port 124 by which the device 100 can be connected to an external power supply using a USB cable. In other embodiments, other suitable power input ports can be used, or the power supply input can be hardwired to the control board such that the device 100 is supplied with a power cable extending therefrom.

The sensor board 202 is connected to the control board 204 by a first set of spacers such as columnar spacers 208 to form a stacked circuit board structure 200 wherein the sensor board 202 and control board 204 form a stacked arrangement with a gap 218 between the two defined by the spacers 208.

When contained within the housing, the sensor board 202 is closer to the front surface 102 of the housing so that the respiratory-related sensors 110 are positioned behind the respective openings in the front surface 102, and the radar sensor is positioned behind the front surface 102, and the control board 204 is closer to the back surface 120 of the housing.

The control board 204 can be attached to the back surface 120, i.e. the separate back panel, by a second set of spacers such as columnar spacers 210, and held in place by a suitable attachment (for example a plurality of screws) 130 such that the sensor board 202 and control board 204 are securely held within the housing.

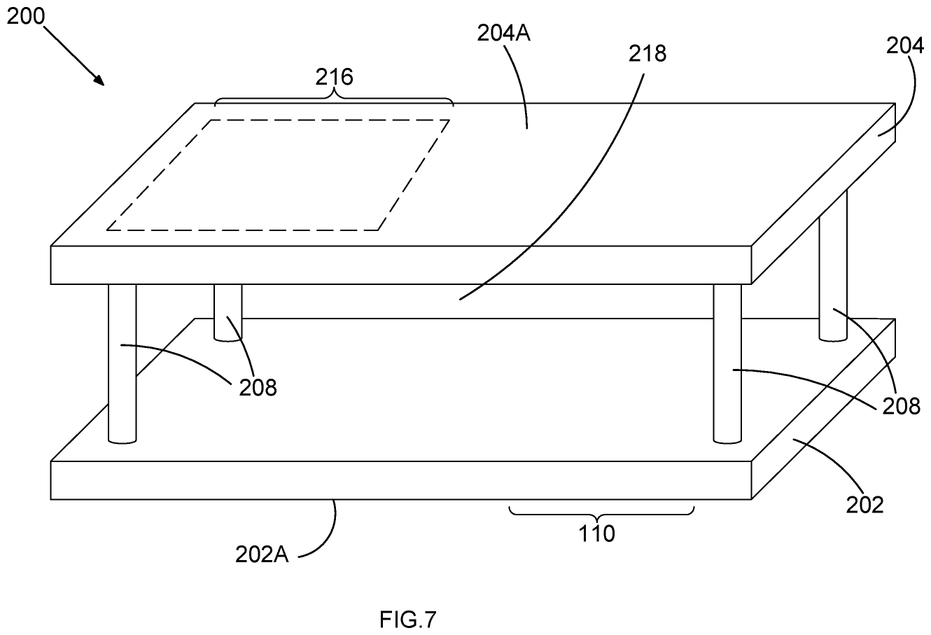

Some components of the control board 204, such as the processor for example, can generate substantial heat when carrying out their operation. Separating such substantially heat-generating components 216 of the control board 204 from the respiratory-related sensors of the sensor board, particularly the environmental sensors such as the temperature sensor, can inhibit local heating from the substantially heat-generating components 216 affecting the temperature measurements. As can be seen in the conceptual diagram of FIG. 7, the respiratory-related sensors 110 are arranged on the sensor board 202 to face away from the control board 204. Likewise, the substantially heat-generating components 216 of the control board 204 are arranged on the control board 204 on a side 204A that faces away from the sensor board 202. That is, the respiratory-related sensors 110 and the substantially heat generating-components 216 of the controller face in opposite directions. The respiratory-related sensors 110 face toward the inner side of the front surface 102 of the housing, and the substantially heat-generating components 216 of the control board face toward the inner side of the back surface 120 of the housing. Furthermore, the respiratory-related sensors 110 are arranged at an end portion of the sensor board 202 opposite to an end portion of the control board 204 at which the substantially heat-generating components 216 of the controller are located (such as the processor). In the example of FIGS. 6A and 6b, the respiratory-related sensors 110 are arranged at a right-most portion of the sensor board 202 (for alignment with the respective openings in the front surface 102 of the housing) and the substantially heat-generating components 216 of the control board 204 are arranged at a left-most portion of the control board 204. The skilled person will however readily understand that the respiratory-related sensors 110 can alternatively be arranged at the left-most portion of the sensor board 202 (with the openings in the front surface 102 of the housing respectively moved) and the heat-generating components can be arranged at a right-most portion of the control board 204. Indeed, any arrangement which utilises a distal separation between the respiratory-related sensors 110 on the sensor board 202 and the substantially heat-generating components 216 on the control board 204 can be used. In this way, a separation between the heat generating components of the control board 204 and the temperature sensor of the respiratory-related sensors 110 on the sensor board 202 is achieved. This provides a thermal isolation that inhibits localised heating from the processor (or other substantially heat-generating components) affecting the temperature measurements recorded by the temperature sensor by convection. This thermal isolation is further enhanced by positioning a thermally insulating material 212, such as thermally insulating foam, in the gap 218 between the control board 204 and the sensor board 202 to inhibit thermal conduction and radiation from the substantially heat-generating components 216 to the temperature sensor.

A radar module 214 comprising the radar sensor is mounted to the sensor board 202 so as to be arranged behind the front surface 102. The radar module 214 can be mounted substantially centrally on the sensor board 202, and in the gap between the sensor board 202 and the control board 204.

By facing the substantially heat-generating components 216 of the control board 204 toward the back surface 120 of the housing, and the control board 204 being attached to the back surface 120 of the housing, the control board components are positioned close to the ventilation holes 122 arranged in the back surface 120 of the housing. This allows for any air that is heated by the substantially heat-generating components 216 (such as the processor) to flow out of the ventilation holes 122, rather than causing localised heating within the device 100 that could affect the ambient temperature measurements recorded by the temperature sensor.

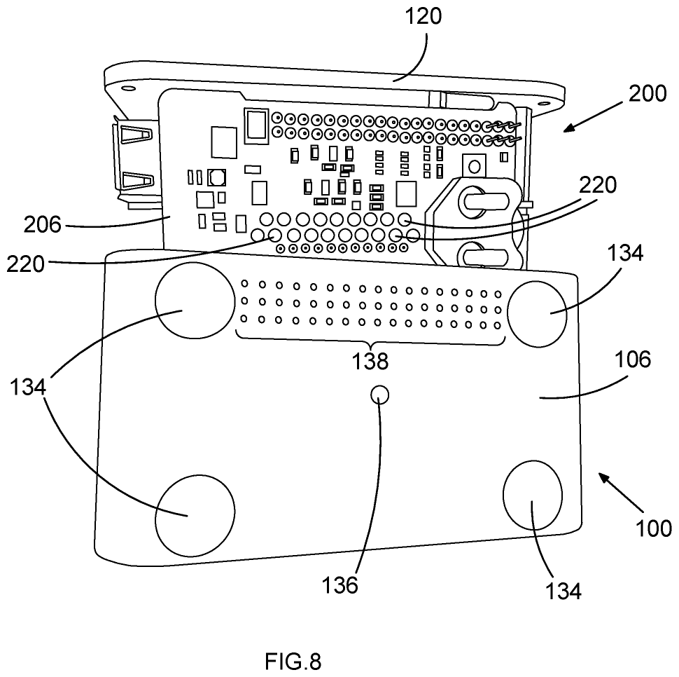

A further circuit board 206 is arranged perpendicular to the sensor board 202 and the control board 204 in the stacked circuit board structure 200. The further circuit board 206 is connected to the sensor board 202 through a right angle connector. A plurality of ventilation holes 220 are included in the further circuit board 206 to allow for a ventilating air flow from the centre of the device 100, through the ventilation holes 220 in the further circuit board 206, and through the corresponding ventilation holes 138 in the bottom side surface 106 of the housing. This is best shown in FIG. 8, which shows a partially exploded diagram of the bottom side surface 106 of the housing, and the stacked circuit board structure 200 removed from the remaining portion of the housing. The further circuit board 206 can include further components associated with and connected to the control board 204, such as power supply modules, a real-time clock RTC component, LED drivers and LEDs (for example the operational information indicator 132 and the device diagnostic indicator 136). Splitting the components associated with the control board 204 between the control board 204 itself and the further circuit board 206 can improve the packing of the circuit boards within the device 100, thereby providing a more compact device 100.

In use, the respiratory monitoring device 100 is arranged such that the front surface 102 and respiratory-related sensors are directed toward a sleeping patient, and more particularly the chest of a sleeping patient. The device 100 can be placed on a nightstand or other suitable surface such that it is approximately level with the sleeping patient and within a close proximity, for example within 1 to 2 metres or closer, or more preferably within 1.5 metres of the patient.

Alternatively, the respiratory monitoring device 100 can be arranged with the front surface 102 and the respiratory-related sensors directed to a person sitting in a chair, working at a desk, or in any other similar position.

To ensure the most accurate measurement and recording of data, the respiratory-related sensors of the device 100 should be directed toward the patient. The layout of the features of the device helps to convey to the user that the side of the device comprising the respiratory-related sensors is the front side, so as to aid the user in knowing to direct this side toward the patient. The visually pleasing nature of the recessed portion 112 of the front surface 102, and the positioning of the respiratory-related sensors 110 therein, and otherwise minimal appearance, when compared to the ventilation holes 122, screw fittings 128, 130 and power input port 124 in the back surface, help to guide the user to direct the front surface 102 toward the patient. By positioning the power input port 124 on the back surface 120, a corresponding power cable attached thereto helps to balance the device 100 and prevent it from tipping forward.

The respiratory monitoring device 100 concurrently measures patient movement data, sound data, and environmental data such as ambient temperature data using the respiratory-related sensors. The respiratory monitoring device 100 measures patient movement data using the radar sensor. In an example the radar sensor may be a one dimensional radar sensor, or a Doppler radar sensor. In particular the movement measurements relate to the movement of the chest of the patient when breathing, hence the importance of the respiratory-related sensors being directed toward the patient. The radar sensor can also be used to monitor movements of the patient, for example when they are sleeping, which can be used in respiratory analysis. The microphone records sound data, and in particular sound data relating to the breathing, coughing of wheezing of the patient. The temperature sensor records the ambient temperature in the vicinity of the device 100 (and consequently the vicinity of the patient).

When the respiratory monitoring device 100 is connected to a power supply, the processor and operating system of the device switch on. The respiratory monitoring device 100 can then remain switched on as long as it is connected to the power supply. In some examples, a power button may be included to switch the device on and off.

The operating system of the respiratory monitoring device 100 controls the recording behaviour and timing of the device. The timing of measurement sessions, that is the times during which the device is to measure and record respiratory-related data, is pre-set in the device and stored in the memory. These pre-set timings can be set by the user or by a clinical study team, for example. The pre-set timings may be adjusted by interfacing with the device through the dataport(s), or by a wireless interface, with an external control device such as a computer or smartphone having associated control software or an application for the respiratory monitoring device 100 stored thereon. In some examples, a clinical study team may remotely adjust the timings when the respiratory monitoring device 100 is connected to a network such as the internet. In this way, the timings for data measurement and recording can be adjusted to suit the patient.

A typical measurement session (i.e. the time period during which the device 100 measures and records data) might correspond to the time during which the patient is asleep or attempting to sleep, for example 9 PM to 7 AM. As noted above, the timing of the measurement session can be adjusted to meet user requirements.

When switched on, the respiratory monitoring device 100 automatically starts and stops the measurements sessions according to the preset measurement session timings.

During the measurement session, when the device 100 is measuring and recording data, the operational information indicator 132 is arranged to convey information in a first way in order to indicate that data is being measured and recorded. For example, when the indicator is a light source 132, such as a one or more mono-colour LEDs, or a multi-colour LED, it can be illuminated in a first colour that is indicative of data being recorded. Alternatively/additionally, the light source 132 can be set to be solidly switched on when data is being measured and recorded, or to flash at a first frequency or in a first pattern that is indicative of data being measured and recorded. The operational indicator 132 can be used to show the monitoring state of the device, for example whether the device is switched on, switched off, or if measurement has been suspended for a predetermined period. The operational indicator can also be used to provide feedback to a user of the device, to show a health warning, or to give instructions to the user to contact an associated research study team or the manufacturer.

The respiratory monitoring device 100 can further include a light sensor. The light sensor is used by the controller to determine the ambient light level in the vicinity of the device 100. The controller can then adjust a brightness level of the operational information indicator light source 132 based upon the ambient light level. For example when the ambient light level is high, i.e. the room is relatively brightly lit, the brightness of the operational information indicator light source 132 can be set to a higher level so that the light emitted is clearly visible to a user. When the ambient light level is low, i.e. the room is relatively dark, the brightness of the operational information indicator light source 132 can be set to a lower level so that the light emitted is less likely to disturb the sleep of a patient whilst still being visible by a separate user to the device 100.

In the example of FIGS. 1 to 8, the operational information indicator light source 132 is arranged at the right-most side surface 108A of the device 100, at a relatively low vertical position. Arranging the operational information indicator light source 132 at a non-front surface, and/or at a relatively low vertical position on the device, is beneficial as it is visible to a user, whilst not being directed toward the patient and thus it is less likely to disturb the sleep of the patient. The skilled person will readily understand that, whilst the operational information indicator light source 132 is arranged on the right-most side surface 108A in the example of FIGS. 1 to 8, it could alternatively be arranged at the left-most side surface 108B, the top side surface 104, the bottom side surface 106 or the back surface 120.

One or more data sets corresponding to the measurements made by the respiratory-related sensors in one or more measurement sessions can be stored on the device 100, relating to one or more nights of sleep of the patient. In an example, the data is stored on a removable memory unit, such as a USB stick inserted into the data port 116. The data is then exportable by removing the USB stick. In another example, the data is stored in built-in storage in the device 100, and can be exported to such a removable memory unit. In another example, the device 100 can be connected to a network such that the data can be exported by a wired connection to the network (for example by an Ethernet cable using an Ethernet data port 118), or by a wireless connection to the network (for example by using a built-in Wi-Fi transmitter/receiver). In another example, the data can be exported to a separate device using a wired or wireless connection (such as a USB cable or a Bluetooth connection, 3G or 4G connection or the like, respectively). The data can be batch exported for multiple measurement sessions; alternatively, the data can be individually exported for a single measurement session. In any of the preceding examples, the data set(s) can be exported to an external processing facility at which the data is analysed, or exported to be analysed by another individual (for example a parent or carer who has access to requisite data processing and analysis software) or another organisation who can process and analyse the data. Information derived from processing the data set(s) can then be fed back to the patient or healthcare provider in the form of useful information relating to their health condition, or directed to a research establishment for research purposes, as appropriate. In other examples, the data can be processed or filtered and analysed within the device using data processing systems executed by a processor of the respiratory monitoring device 100.

When in use, in some situations, a person may enter the room in which data is being recorded by the respiratory monitoring device. For example, if the patient is child, a parent or care provider may enter the room to tend to them if their sleep has been disrupted. The presence of such a third party in the room can interfere with the data recording, and produce artefacts in the measured respiratory data (such as sounds, movements or temperature changes etc.), which would be convoluted in the recorded data through detection by the respiratory-related sensors. Such artefacts can affect the usefulness of the data. Additionally, at some points during the measurement session a user may wish to suspend the measurement and recording of data, for example for privacy reasons or because they expect to be away from the respiratory monitoring device 100 for a period of time. To address these problems, the device 100 includes a user input means 114, such as a button, that pauses the recording of data.

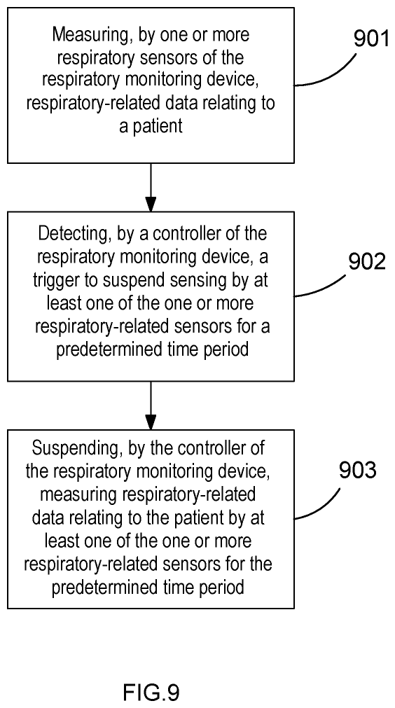

When activated, the user input means 114 triggers the controller to suspend the measurement and recording of data by the respiratory-related sensors, or one or more of the respiratory-related sensors. This suspension is maintained for a predetermined time period. This summarised in the flow chart of FIG. 9. At step 901 the one or more respiratory-related sensors of the respiratory monitoring device measure respiratory-related data relating to a patient. Then, at step 902, the controller of respiratory monitoring device detects a trigger to suspend sensing by at least one of the one or more respiratory-related sensors for a predetermined time period. At step 903, in response to detecting the trigger, the controller of the respiratory monitoring device suspends measuring respiratory-related data relating to the patient by at least one of the one or more respiratory-related sensors for the predetermined time period.

As previously described, the user input means 114 can be a pushbutton. In other examples the user input means can be a capacitive button, a touch screen, or any other suitable means by which a user can trigger the controller to suspend the measurement and recording of data.

In a first example of a predetermined suspension time period, the predetermined time can be a fixed period of time, such as one hour (the skilled person will however readily understand that the fixed time need not be one hour, but could be any other suitable length of time). Following the expiration of the fixed time period, the measurement and recording of data is resumed until the end of the measurement session. In this way, when the patient is a child, for example, a parent or care provider can enter the room, trigger the suspension of the measurement and recording of data by one or more of the respiratory-related sensors by actuating the user input means 114 (e.g. pressing the pushbutton), and tend to the child without any associated noise, movements or temperature changes being convoluted in the data of the measurement session.

In a second example a predetermined suspension time can be the remaining time of the measurement session. That is, when the user input means 114 is actuated, the measurement and recording of data is suspended until the end of the measurement session. In other words, the predetermined suspension time is the difference between the point in time that the user input means 114 is actuated and the scheduled end of time of the measurement session. In this way, when the entire remainder of the measurement session is likely to be disrupted, measurement and recording of data by one or more of the respiratory-related sensors for the remainder of the session can be suspended so that a bad measurement session data set is not included with useful measurement session data sets, or to address privacy concerns, or to avoid recording unnecessary data if the patient is not present.

These suspension times can be customisable, for example through an interface with the device by a user, or by the manufacturer. The suspension times may be adjusted by interfacing with the device through the dataport(s), or by a wireless interface, with an external control device such as a computer or smartphone having associated control software or an application for the respiratory monitoring device 100 stored thereon.

The user input means 114 can be arranged such that it can be actuated in a number of different manners in order to trigger different predetermined suspension time periods.

In the case of a pushbutton 114, in a first manner of actuating the pushbutton 114, the pushbutton can be depressed for a first time duration; that is, a ‘press and release’ of the button 114 (for example, not holding the button down for more than 3 seconds) can be used to trigger a first predetermined suspension time period (such as the first example of the predetermined suspension time period described above). In a second manner of actuating the pushbutton 114, the pushbutton can be depressed for a second time duration longer than the first time duration; that is, a ‘press and hold and release’ action, i.e. pressing and holding the button 114 for a period of time (for example, holding the button down for 5 seconds or more) before releasing the button 114 can be used to trigger a second predetermined suspension time period (such as the second example of the predetermined suspension time period as described above). In a specific example, when the pushbutton is depressed for 0.5 to 3 seconds the measurement and recording of data is suspended for 1 hour, and when the pushbutton is depressed for 5 to 10 seconds the measurement and recording of data is suspended for the entire night (until the end of the measurement session). The skilled person would readily understand that in an alternative, a ‘press and release’ of the button 114 could suspend the measurement and recording of data for the second predetermined time period, and a ‘press and hold and release’ action on the button 114 could suspend the measurement and recording of data for the first time period. The skilled person would also readily understand that a ‘press and release’ or a ‘press and hold and release’ action on the button 114 could suspend the measurement and recording of data for any suitable time period. The first and second time durations of the press for a ‘press and release’ and a ‘press and hold and release’ can be customisable, for example through an interface with the device by a user, or by the manufacturer. The duration settings of the ‘press and release’ and ‘press and hold and release’ may be adjusted by interfacing with the device through the dataport(s), or by a wireless interface, with an external control device such as a computer or smartphone having associated control software or an application for the respiratory monitoring device 100 stored thereon.

In an alternative, the pushbutton 114 can have two depression states corresponding to two actuation manners of the pushbutton 114. The first actuation manner uses a first depression state in which the button 114 is partially depressed through its working distance, and the second actuation manner uses a second depression state in which the button 114 is fully depressed through its working distance. The button 114 can be provided with a haptic feedback to indicate when the button 114 has been partially depressed and fully depressed. This allows the user to identify whether they have partially depressed the button 114 in the first actuation manner, or fully depressed the button 114 in the second actuation manner.

Partially depressing the button 114, i.e. actuating the button 114 in the first manner, can suspend the measurement and recording of data for the first predetermined time period (such as the first example of the predetermined suspension time period described above). Fully depressing the button 114, i.e. actuating the button in the second manner, can suspend the measurement and recording of data for the second predetermined time period (such as the second example of the predetermined suspension time period as described above). The skilled person would readily understand that in an alternative, partially depressing the button 114 could suspend the measurement and recording of data for the second predetermined time period, and fully depressing the button 114 could suspend the measurement and recording of data for the first time period. The skilled person would also readily understand that partially or fully depressing the button 114 could suspend the measurement and recording of data for any suitable time period.

The indicator that conveys operational information 132 can be used to indicate that the measurement and recording of data has been suspended. For example, in the case of the indicator being a light source 132, when data is being measured and recorded the light source can be set to convey information in the first way as described above (such as by illuminating the light source 132 in a first colour, the light source being solidly switched on, or flashing in first pattern or at a first frequency).

When the first predetermined suspension time period has been triggered the operational information indicator 132 can be set to convey information in a second way. For example, the light source 132 can be illuminated in a second colour different to the first colour, and/or programmed to flash in a second pattern/frequency that is different to the first pattern/frequency.

When the second predetermined measurement suspension time period has been triggered, the operational information indicator 132 can be set to convey information in a third way. For example, the light source 132 can be illuminated in the second colour, or a third colour different to the first and second colours, and/or programmed to flash in a pattern/frequency that is different to the first and second pattern/frequency. In another example, the third colour can be the same as the second colour, and/or the third pattern/frequency can be the same as the second pattern frequency, such that the operational indicator 132 of a suspension is the same for all types of the predetermined suspension time period.

The respiratory monitoring device 100 may further comprise a user interface, such as one or more buttons and/or a screen or touch screen, to allow the user to input information such as another person being present in the room, entering self-reported symptoms, keeping track of medication use, and triggering an emergency alarm (for example to healthcare providers or family).

It will be readily understood to the skilled person that the preceding embodiments are not limiting; features of each embodiment may be incorporated into the other embodiments as appropriate.

The processing steps described herein carried out by the device may be stored in a non-transitory computer-readable medium, or storage, associated with the electronic device. A computer-readable medium can include non-volatile media and volatile media. Volatile media can include semiconductor memories and dynamic memories, amongst others. Non-volatile media can include optical disks and magnetic disks, amongst others.

1.-7. (canceled)

8. A respiratory monitoring device configured to measure respiratory related data, the respiratory monitoring device comprising:
a controller;
one or more respiratory-related sensors; and
an input means;
wherein the input means is arranged to trigger the controller to suspend sensing by at least one of the one or more respiratory-related sensors for one or more predetermined time periods.

9. The respiratory monitoring device of claim 8, wherein at least one predetermined time period of the one or more predetermined time periods is a fixed time period.

10. The respiratory monitoring device of claim 8, wherein at least one predetermined period of the one or more predetermined time periods is a time period between the respiratory-related sensors being suspended and a scheduled end time for a respiratory data measurement session during which the suspension is triggered.

11. The respiratory monitoring device of claim 8, wherein the input means comprises a user-operable button.

12. The respiratory monitoring device of claim 11, wherein the user-operable button is actuatable in a first manner to trigger the device to suspend sensing for a first predetermined time period of the one or more predetermined time periods, and in a second manner to trigger the device to suspend sensing for a second predetermined time period of the one or more predetermined time periods, the second predetermined time period different to the first time period.

13. The respiratory monitoring device of claim 12, wherein in the first manner the user-operable button is actuated for a first actuation time, and in the second manner the user-operable button is actuated for a second actuation time.

14. The respiratory monitoring device of claim 12, wherein the user-operable button is depressable in two stages such in the first manner the user-operable button is partially depressed, and in the second manner the user-operable button is fully depressed.

15. The respiratory monitoring device of claim 14, wherein the user-operable button is arranged to provide a haptic feedback indicating that the user-operable button has been partially depressed and fully depressed.

16. The respiratory monitoring device of claim 8, further comprising an indicator arranged to indicate when the device has been triggered to suspend sensing.

17. The respiratory monitoring device of claim 16, wherein the indicator is arranged to indicate information relating to the predetermined time period for which sensing is suspended.

18. The respiratory monitoring device of claim 17, wherein the indicator comprises a light source.

19. The respiratory monitoring device of claim 18, wherein the light source is arranged at a different side of the respiratory monitoring device to a side at which the one or more respiratory-related sensors are arranged.

20. The respiratory monitoring device of claim 18, further comprising an ambient light sensor, and wherein a brightness of the light source is adjusted in accordance with an ambient light level detected by the ambient light sensor.

21. A respiratory monitoring method at a respiratory monitoring device, the method comprising:
measuring, by one or more respiratory-related sensors of the respiratory monitoring device, respiratory-related data relating to a person;
detecting, by a controller of the respiratory monitoring device, a trigger to suspend sensing by at least one of the one or more respiratory-related sensors for a predetermined time period;
suspending, by the controller of the respiratory monitoring device, measuring respiratory-related data relating to the person by at least one of the one or more respiratory-related sensors for the predetermined time period.

22. A non-transitory computer-readable medium storing instructions that, when executed by one or more processors of a respiratory monitoring device, cause the one or more processors to carry out the steps of:
measuring, by one or more respiratory-related sensors of the respiratory monitoring device, respiratory-related data relating to a person;
detecting, by a controller of the respiratory monitoring device, a trigger to suspend sensing by at least one of the one or more respiratory-related sensors for a predetermined time period;
suspending, by the controller of the respiratory monitoring device, measuring respiratory-related data relating to the person by at least one of the one or more respiratory-related sensors for the predetermined time period.

23-34. (canceled)

35. The respiratory monitoring device of claim 8, wherein the one or more respiratory-related sensors are arranged to measure at least one of: movement proximal to the respiratory monitoring device, sound proximal to the respiratory monitoring device, or environmental data proximal to the respiratory monitoring device.

36. The respiratory monitoring device of claim 35, wherein the respiratory monitoring device comprises at least one of:
a radar sensor to measure movement proximal to the respiratory monitoring device;
a microphone to measure acoustic data proximal to the respiratory monitoring device;
a temperature sensor arranged to measure the ambient temperature proximal to the respiratory monitoring device; or
a humidity sensor arranged to measure humidity proximal to the respiratory monitoring device.
